SEQUENCE LISTING (28 sequences)

SEQ ID NO: 1 - DNA, 12 bp, Artificial Sequence
  Primer Oligonucleotide
ttataatgtttt

SEQ ID NO: 2 - DNA, 12 bp, Artificial Sequence
  Primer Oligonucleotide
taatatactaat

SEQ ID NO: 3 - DNA, 18 bp, Homo sapiens
tgtcatcctgttgtcatg

SEQ ID NO: 4 - DNA, 18 bp, Artificial Sequence
  Primer Oligonucleotide
tgttattttgttgtttag

SEQ ID NO: 5 - DNA, 18 bp, Artificial Sequence
  Primer Oligonucleotide
tatcatcctattatgata

SEQ ID NO: 6 - DNA, 14 bp, Artificial Sequence
  Adaptor Oligonucleotide
tgtcatcctgttgt

SEQ ID NO: 7 - DNA, 18 bp, Artificial Sequence
  Primer Oligonucleotide
tgttattttgttgttatg

SEQ ID NO: 8 - DNA, 18 bp, Artificial Sequence
  Primer Oligonucleotide
tatcatcctattatcata

SEQ ID NO: 9 - DNA, 12 bp, Homo sapiens
  modified_base (4)..(4): Methylcytosine
dcdcgcatgddd

SEQ ID NO: 10 - DNA, 15 bp, Homo sapiens
  modified_base (8)..(8): Methylcytosine
dddcatgcgdgcatg

SEQ ID NO: 11 - DNA, 26 bp, Artificial Sequence
  Oligonucleotide
tgtcatcctgttgtcatgcdcgcatg

SEQ ID NO: 12 - DNA, 26 bp, Artificial Sequence
  Oligonucleotide
tgtcatcctgttgtcatgcgdgcatg

SEQ ID NO: 13 - DNA, 40 bp, Artificial Sequence
  Oligonucleotide
tgtcatcctgttgtcatgcdcgcatgacaacaggatgaca

SEQ ID NO: 14 - DNA, 40 bp, Artificial Sequence
  Oligonucleotide
tgtcatcctgttgtcatgcgdgcatgacaacaggatgaca

SEQ ID NO: 15 - DNA, 40 bp, Artificial Sequence; identical to SEQ ID NO: 13
  Oligonucleotide

SEQ ID NO: 16 - DNA, 40 bp, Artificial Sequence; identical to SEQ ID NO: 14
  Oligonucleotide

SEQ ID NO: 17 - DNA, 40 bp, Artificial Sequence; identical to SEQ ID NO: 13
  Oligonucleotide

SEQ ID NO: 18 - DNA, 40 bp, Artificial Sequence; identical to SEQ ID NO: 14
  Oligonucleotide

SEQ ID NO: 19 - DNA, 40 bp, Artificial Sequence
  chemically treated DNA-oligonucleotide
uguuauuuuguuguuaugunuguaugauaauaggaugaua

SEQ ID NO: 20 - DNA, 40 bp, Artificial Sequence
  chemicially treated DNA-oligonucleotide
uguuauuuuguuguuaugugnguaugauaauaggaugaua

SEQ ID NO: 21 - DNA, 40 bp, Artificial Sequence
  Oligonucleotide
tgttattttgttgttatgtdtgtatgataataggatgata

SEQ ID NO: 22 - DNA, 40 bp, Artificial Sequence
  Oligonucleotide
tatcatcctattatcatacadacataacaacaaaataaca

SEQ ID NO: 23 - DNA, 40 bp, Artificial Sequence; identical to SEQ ID NO: 13
  Oligonucleotide

SEQ ID NO: 24 - DNA, 41 bp, Artificial Sequence
  Oligonucleotide
tgtcatcctgttgtcatgncgdgcatgacaacaggatgaca

SEQ ID NO: 25 - DNA, 40 bp, Artificial Sequence
  chemically treated DNA-oligomer
uguuauuuuguuguuaugudcguaugauaauaggaugaua

SEQ ID NO: 26 - DNA, 40 bp, Artificial Sequence
  chemically treated DNA-oligonucleotide
uguuauuuuguuguuaugcgdguaugauaauaggaugaua

SEQ ID NO: 27 - DNA, 40 bp, Artificial Sequence
  Oligonucleotide
tgttattttgttgttatgtdcgtatgataataggatgata

SEQ ID NO: 28 - DNA, 40 bp, Artificial Sequence
  Oligonucleotide
tatcatcctattatcatacgdacataacaacaaaataaca

(19) United States
(12) Patent Application Publication
(10) Pub. No.: US 2005/0019762 A1
(43) Pub. Date: Jan. 27, 2005

(54) Method for quantifying cytosine methylations in genomic dna that is amplified in a complex manner

(76) Inventor: Alexander Olek, Berlin (DE)

Correspondence Address: KRIEGSMAN & KRIEGSMAN, 665 FRANKLIN STREET, FRAMINGHAM, MA, 01702, US

(21) Appl. No.: 10/433,742

(22) PCT Filed: Dec. 5, 2001
(86) PCT No.: PCT/DE01/04617

(30) Foreign Application Priority Data
Dec. 6, 2000 (DE) 10061348.9

(51) Int. Cl. C12Q 1/68; C12P 19/34

(52) U.S. Cl. 435/6; 435/91.2

(57) ABSTRACT

A method is described for preparing demethylated DNA as reference material for the analysis of cytosine methylations in genomic DNA samples with the use of complex amplification.

The invention concerns a method for the quantification of cytosine methylations of a genomic DNA sample with unknown methylation status by comparison with a demethylated reference DNA.

5-Methylcytosine is the most frequent covalently modified base in the DNA of eukaryotic cells. For example, it plays a role in the regulation of transcription, genomic imprinting and in tumorigenesis. The identification of 5-methylcytosine as a component of genetic information is thus of considerable interest. 5-Methylcytosine positions, however, cannot be identified by sequencing, since 5-methylcytosine has the same base-pairing behavior as cytosine. In addition, in the case of a PCR amplification, the epigenetic information which is borne by the 5-methylcytosines is completely lost.

The amplification of DNA by means of PCR is state of the art.

Several methods are known which solve these problems. For the most part, a chemical reaction or enzymatic treatment of the genomic DNA is conducted, as a consequence of which cytosine bases can be distinguished from methylcytosine bases. One familiar method is the conversion of genomic DNA with bisulfite, which leads to a conversion of cytosine bases to uracil in two steps after alkaline hydrolysis (Shapiro, R., Cohen, B., and Servis, R. Nature 227, 1047 (1970)). 5-Methylcytosine remains unchanged under these conditions. The conversion of C to U leads to a change of the base sequence from which the original 5-methylcytosines can now be determined by sequencing.

The modification of the genomic base cytosine to 5-methylcytosine represents up to today the most important and best-investigated epigenetic parameter. Nevertheless, even though there are now methods for determining complete genotypes of cells and individuals, there are still no comparable approaches for also generating and extensively evaluating epigenotypic information.

Demethylated DNA is used in the prior art in numerous methods for quantification of DNA methylation. The following are named as representative techniques: “Rapid quantitation of methylation differences at specific sites using methylation-sensitive single nucleotide primer extension” (Gonzalgo, M. L., Jones P. A. Nucleic Acids Res. 25, 2529 (1997)) and “Detection and measurement of PCR bias in quantitative methylation analysis of bisulphite-treated DNA” (Warnecke, P. M., Stirzaker, C., Melki, J. R., Douglas, S. M., Paul, C. L., Clark, S. J. [Journal] 25, 4422 (1997)). This demethylated DNA is obtained, e.g., from cells which are demethylated in the target sequence, or from cells which lack the enzyme, DNA methyltransferase.

On the other hand, it is easily possible to produce demethylated DNA fragments by means of PCR, since the methylation information becomes lost during the amplification, i.e., cytosine is always incorporated instead of methylcytosine, if dCTP is utilized as usual in the polymerase reaction.

If one would like to investigate cytosine methylation, however, as mentioned above, by means of the bisulfite method, in which all unmethylated cytosine bases are converted to uracil and finally to thymine, then for the quantification, a reference DNA must be produced, which contains thymine, instead of all methylated and unmethylated cytosines. This DNA then serves as reference material for a methylation state of 0%. This DNA can be obtained in the simplest way, for the analysis of individual fragments, by first conducting a PCR of the genomic DNA sample in a first amplification and thus the desired fragment is produced, which then essentially no longer has any methylation. The bisulfite treatment is subsequently conducted and the fragment in question is now amplified for the second time with appropriate, but different primers.

This method is not suitable, however, for conducting complex amplifications, which will provide many fragments simultaneously for methylation detection. The problem arises here that it is difficult to ensure that the first amplification also substantially produces the fragments which will be amplified in the second PCR after the bisulfite reaction. This is essential, however, since the sample to be investigated, for whose methylation analysis the reference DNA is produced, is usually amplified exclusively after the bisulfite treatment. Therefore, in a complex PCR reaction, the reference and the sample can no longer be compared, since they potentially contain different fragments.

Presentation of the Problem

The present invention provides a method for the analysis of cytosine methylations in genomic DNA samples, for the purpose of which, DNA with a methylation degree of 0% is produced as reference material. It is thus possible for the first time to produce a substantially unmethylated reference DNA for complex amplifications.

### DESCRIPTION

The present invention describes a method for providing demethylated DNA as reference material for the analysis of cytosine methylations in genomic DNA samples with the use of complex amplifications. The following method steps are conducted individually for this:

a) A genomic DNA sample is amplified with primers, which are either very short or degenerate oligonucleotides or oligonucleotides complementary to adaptors. In the second case, prior to the amplification, the sample is cleaved with a restriction enzyme, and the adaptors, which are understood to be short nucleotide fragments of known sequence, are ligated to the ends of the DNA fragments that are formed.

The amplificates are chemically treated in such a way that cytosine bases that are unmethylated at the 5-position are converted to uracil, thymine or another base unlike cytosine in hybridization behavior, while the 5-methylcytosine bases remain essentially unchanged. This is understood in the following as chemical pretreatment.

The chemically pretreated amplificates are again amplified. Either several specifically hybridizing oligonucleotides or oligonucleotides complementary to adaptors are used as primers for this purpose. The chemical pretreatment is also conducted for the latter case.

b) A genomic DNA sample to be investigated is cleaved by means of a restriction enzyme. Adaptors are ligated to the ends of the DNA fragments and the sample is then divided. The first portion of the sample is amplified with primer oligonucleotides, which are complementary to the adaptors. In contrast, the second portion of the sample is not amplified.

The two parts of the sample are chemically pretreated and then amplified separately, whereby primer oligonucleotides, which are complementary to the adaptors, are used. The two portions of the sample are then analyzed. The first portion of the sample thus supplies the reference value for a methylation degree of 0%. In contrast, the second portion of the sample supplies the measurement value that essentially corresponds to the degree of methylation in the original genomic DNA sample.

The genomic DNA to be analyzed is obtained preferably from the usual sources for DNA, such as, e.g., cell lines, blood, sputum, stool, urine, cerebrospinal fluid, tissue embedded in paraffin, for example, tissue from eyes, intestine, kidney, brain, heart, prostate, lung, breast, liver, skin or bone marrow, histological slides and all possible combinations thereof.

Preferably, the above-described treatment of genomic DNA with bisulfite (hydrogen sulfite, disulfite) and subsequent alkaline hydrolysis, which converts unmethylated cytosine nuleobases to uracil, is used for this purpose.

In a particularly preferred variant of the method, the polymerase chain reaction (PCR) is used for the amplification. A heat-stable DNA polymerase is preferably used for the polymerase chain reaction. The amplification of several identical or several different DNA segments is preferably conducted in one reaction vessel.

The following are preferably used as restriction endonucleases: RsaI, DpnI, DpnII, MseI, Sau3AI, AluI, NlaIII, HaeIII, BfaI, Tsp509I, BstUI or MboI.

After the chemical treatment, the amplificates are separated from the reagents and other components of the reaction mixture by binding to a solid phase or to a gel and subsequent washing steps.

The reagents and the other components of the reaction mixture are preferably then diluted in such a way that they are no longer troublesome in the subsequent amplification, but the concentration of the treated amplificate is still sufficient for the second amplification.

The demethylated reference DNA which is produced is most preferably analyzed in the same way as a sample DNA to be investigated. This reference DNA supplies in the analysis the reference value for a methylation degree of 0%. Preferably, a DNA which is methylated enzymatically and which is treated in the same way as the sample DNA in the following [steps], additionally serves as a reference for a methylation degree of 100%.

The following examples explain the invention:

### EXAMPLE 1a

### Preparation of a Demethylated Reference DNA by Means of Multiplex PCR

The following example refers to the preparation of a down-methylated DNA sample, which serves as the reference in comparison to an unknown methylated DNA. A genomic DNA sample, which was digested in this case with the restriction enzyme, MssI, is used. Then (1-40 ng) of the cleaved DNA are amplified by a preamplification, by conducting a DOP-PCR (degenerate oligonucleotide primed polymerase chain reaction) according to the method of Nelson (V. G. Cheung, S. F. Nelson, PNAS 93, 1476-1479, 1996) with the genomic primer oligonucleotide 5′-CCGACTCGAGNNNNNNATGTG G-3′. The method particularly serves for the purpose of preamplifying very small quantities of genomic DNA, in order to permit a multiple genetic analysis from 2-15 μg (200-1000 bp). All methylcytosines are treated as cytosine bases in the amplification.

Reaction batch (50 μl):
1 μl (1-40 ng) DNA
2 μl (2 μM) DOP Primer (5′-CCGACTCGAGNNNNNNATGTG G-3′)
5 μl (200 μM) dNTP's (Fermentas)
5 μl PCR buffer (10×, 15 mM MgCl₂) (Qiagen)
0.5 μl (2.5 U) Taq Polymerase (HotstarTaq, Qiagen)
36.5 μl water (for molecular biology, Fluka)

The PCR reaction is conducted in the Master Cycler Gradient (Eppendorf, Hamburg) with the following program.

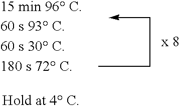

The PCR sample is diluted with water (1:10-1:100) and 1 ul of the diluted material is chemically converted with hydrogen sulfite (=bisulfite, disulfite). The DNA is first thermally denatured and then reacted with hydrogen sulfite (=bisulfite, disulfite), a radical trap and a denaturing reagent, and is incubated for a relatively long time at elevated temperature. The bisulfite reaction leads to the conversion of all cytosine bases to uracil. In order to purify the bisulfited DNA, it is bound to a reversed-phase C18 solid phase and is freed of chemicals by washing with a suitable buffer solution. Then the DNA is eluted with a polar solvent, such as, e.g., acetonitrile or water and concentrated to a smaller volume. Alkaline hydrolysis of the fragments treated with hydrogen sulfite (=bisulfit, disulfite) is conducted for 20 min at 96° C. under basic conditions directly prior to the specific amplification. Preferably 1-500 different primer oligonucleotides which do not contain wobble base pairings are utilized in this reaction. In this Example, the specific amplification is conducted with 128 primer oligonucleotides, wherein at least 64 primer oligonucleotides are labeled with Cy5 (Amersham Pharmacia). One primer oligonucleotide of a primer pair is labeled each time.

Reaction batch, multiplex PCR (25 μl)
1 μl DNA treated with hydrogen sulfite
2.5 μl PCR buffer (10×, Qiagen)
0.6 μl Primer oligonucleotide mixture (128 primer oligonucleotides, 64 of which are labeled with Cy5, 0.78 pmol/μl of each)
0.8 μl dNTPs (25 mM per dNTP, Gibco-BRL)
3 μl MgCl₂ (15 mM)
4.5 μl water (for molecular biology, Fluka)
12.5 μl Tris-HCl (pH 9.5; 100 mM)
0.2 μl Polymerase (1 unit) (HotstarTaq, Qiagen)

The PCR reaction is conducted in the Master Cycler Gradient (Eppendorf, Hamburg) with the following program.

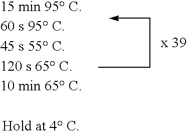

The PCR amplificates produced were analyzed by agarose gel electrophoresis (1.5% agarose in 0.5×TBE buffer, Sambrook et al.). For this, 4 μl of the PCR batch are subjected to gel electrophoresis. Under the given conditions, 64 genes are successfully amplified simultaneously.

### EXAMPLE 1B

### Preparation of an Unknown Methylated DNA Sample by Means of Multiplex PCR

The following Example 1b concerns the preparation of an unknown methylated DNA sample, which is compared with the down-methylated reference DNA from Example 1a. A genomic DNA sample is used, which was cleaved in this case with the restriction enzyme, MssI. The sample is then reacted with hydrogen sulfite (=bisulfite, disulfite). Then one can proceed according to 2 different methods. The first method (Olek et al., Nucl. Acids Res. 1996, 24, 5064-5066) is a conversion with hydrogen sulfite and a radical trap, wherein the DNA is embedded in agarose. The desulfonation of the DNA is also conducted in agarose. The DNA is used in this case without further purification operations in a preamplification (PEP=primer extension preamplification). Alternatively, the DNA can also be chemically converted without agarose matrix employing hydrogen sulfite (=bisulfite, disulfite) and a radical trap at elevated temperature. An organic reagent, which supports the denaturation, is added, and the batch is incubated at elevated temperature. All cytosine bases are converted to uracil in both methods by the treatment with hydrogen sulfite, whereas methylcytosines remain the same. In order to purify the bisulfited DNA without agarose matrix, it is bound to a reversed-phase C18 solid phase and is freed of chemicals by washing with a suitable buffer solution. Then the DNA is eluted with a polar solvent, such as, e.g., acetonitrile and water and concentrated to a smaller volume. The preamplification of the DNA treated with hydrogen sulfite is conducted with degenerate primer oligonucleotidea (5′-TTATAATGTTTT and 5′-TAATACTAAT).

Reaction batch (20 μl):
1 μl bisulphite DNA (0.2-1 ng)
2 μl reaction buffer (10×, Qiagen)
2 μl dNTP's (10 mM per dNTP, Fermentas)
1 μl Primer (TTATAATGTTTT) 25 pmol
1 μl Primer (TAATATACTAAT) 25 pmol
0.2 μl polymerase (1 unit) (HotstarTaq, Qiagen)
12.8 μl water (for molecular biology, Fluka)

The following amplification with Cy5-labeled bisulfite-specific primer oligonucleotides is conducted with the 128 primer oligonucleotides described in Example 1a, whereby the same primer oligonucleotide is labeled with Cy5. The amplificates are also subjected to an agarose gel electrophoresis for analysis.

### EXAMPLE 1c

### Comparison of the Unknown Methylated DNA Sample with the Down-Methylated Reference DNA

The unknown methylated DNA sample is compared with the down-methylated reference DNA, preferably by hybridization on an oligonucleotide array. Fluorescing points are visible corresponding to position on the array. It happens that specific points show a clearly increased or decreased fluorescence relative to other points and to the reference DNA, as long as the amplificates are present in comparable concentration in the individual samples to be investigated. The intensity of the fluorescent dye Cy5 (635 nm) is measured in the individual amplificates. Techniques for the evaluation of fluorescence measurements are known to the person skilled in the art.

### EXAMPLE 2

### Preparation of Demethylated Reference DNA

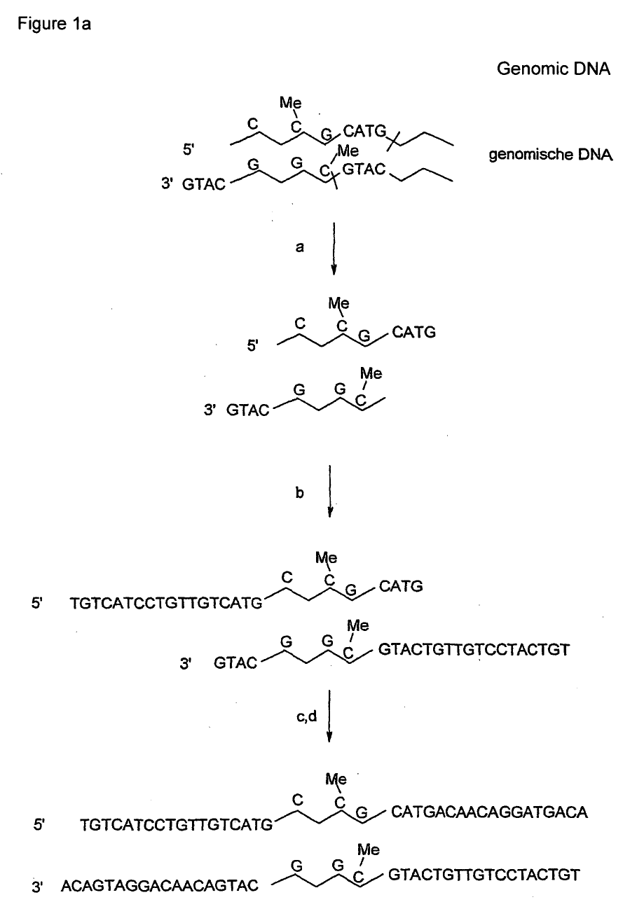

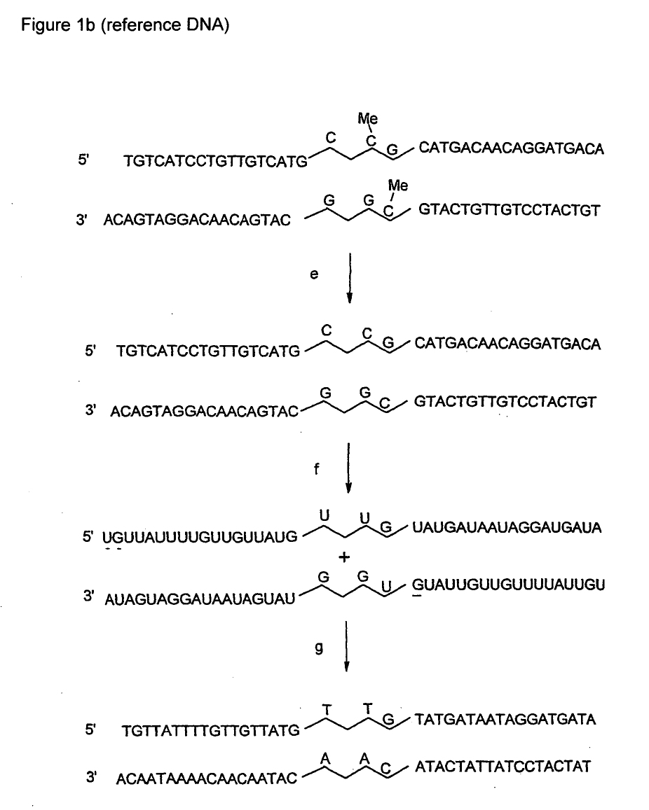

In the first step, a genomic sequence is enzymatically cleaved according to the manufacturer's instructions by addition of a restriction enzyme, here NlaIII (Fermentas), which recognizes the sequence CATG. Thus, fragments of an average 400 bp in size are produced. The cleaved fragments have 3′ overhanging CATG ends and are ligated with the oligomer with the genomic sequence TGTCATCCTGTTGTCATG with the addition of T4-DNA ligase according to standard conditions (Fermentas) at the sequence segments and unligated adaptors are removed according to standard conditions with a purification kit (Qiaquick PCR Purification Kit, Qiagen). Then the single-stranded ends are completed to form the double strand with Klenow enzyme (DNA Polymerase I, Roche Molecular Biochemicals) and dNTP's (FIG. 1a).

If a reference DNA is to be produced, then the procedure is the following: In the following step, the ligated sequence segments are amplified in a PCR reaction with the addition of primer oligonucleotides with the sequence TGTCATCCTGTTGTCATG and with a heat-stable DNA polymerase. The PCR reaction is conducted in the Master Cycler Gradient (Eppendorf, Hamburg) with the following parameters: Denaturation: 15 minutes (min) at 96° C., [and] the following cycles are repeated 45 times: 60 seconds (sec) at 96° C., 45 sec at 51° C., 60 sec at 72° C. and subsequent incubation for 10 minutes at 72° C.

In the next step, the DNA is treated with the use of bisulfite (hydrogen sulfite, disulfite) in such a way that all of the unmethylated cytosines at the 5-position of the base are modified such that a base that is different in its base-pairing behavior is formed, while the cytosines that are methylated in the 5-position remain unchanged. If 1.7 M bisulfite solution is used for the reaction, then an addition occurs at the unmethylated cytosine bases. Also, a denaturing reagent or solvent as well as a radical trap must be present. A subsequent alkaline hydrolysis then leads to the conversion of unmethylated cytosine nucleobases to uracil. This converted DNA serves for the detection of methylated cytosines. In the last step of the method, the treated DNA sample is diluted with water or an aqueous solution. A desulfonation of the DNA (20 min, 96° C.) at pH 9 is then preferably conducted. In the last step of the method, the DNA sample is amplified with the primers now complementary to the bisulfite-treated DNA, again in a polymerase chain reaction. The PCR reaction is conducted in the Master Cycler Gradient (Eppendorf, Hamburg) with the following parameters: Denaturation: 15 minutes (min) at 96° C., [and] the following cycles are repeated 45 times: 60 seconds (sec) at 96° C., 45 sec at 42° C., 60 sec at 72° C. and subsequent incubation for 10 minutes at 72° C. (FIG. 1b).

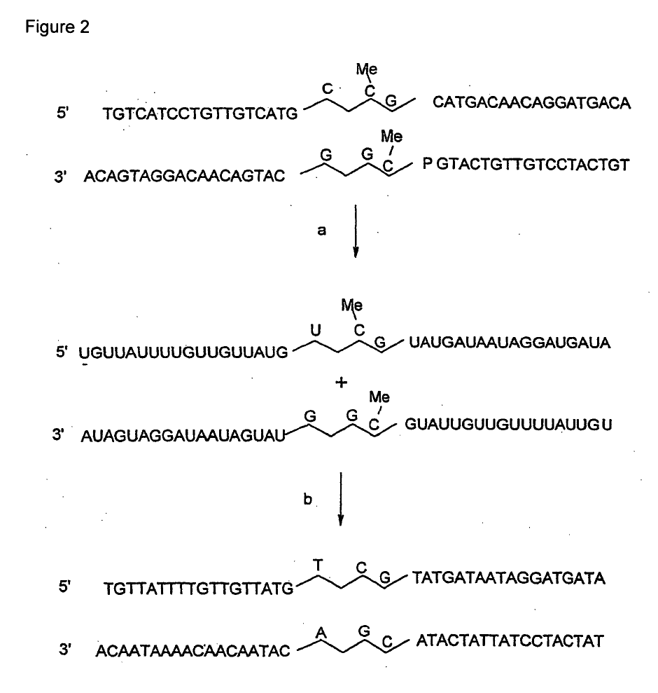

If a DNA with unknown methylation state is to be investigated (FIG. 2), then the cleaved DNA ligated with adaptors (FIG. 1a) is to be treated with bisulfite. After the bisulfite treatment, a PCR is conducted, whereby primer oligonucleotides with the sequences TGTTATTTTGTTGTTTAG and TATCATCCTATTATGATA are used. The PCR reaction is conducted in the Master Cycler Gradient (Eppendorf, Hamburg) with the following parameters: Denaturation: 15 minutes (min) at 96° C.; [and] the following cycles are repeated 45 times: 60 seconds (sec) at 96° C., 45 sec at 42° C., 60 sec at 72° C. and subsequent incubation for 10 minutes at 72° C.

Both in the case of the down-methylated reference DNA as well as in the case of a DNA with unknown methylation state, the detection of the hybridization product is based on primer oligonucleotides fluorescently labeled with Cy5, which were used for the amplification. A hybridization reaction of the amplified DNA with the oligonucleotide occurs only if a methylated cytosine was present at this site in the bisulfite-treated DNA. Thus the methylation state of the respective cytosine to be investigated decides the hybridization product.

Legends to the figures which follow:

FIG. 1a
a) Restriction enzyme, NlaIII
b) Adaptor 1 5′-TGTCATCCTGTTGT, ligase e.g. T4-DNA
c) Kienow enzyme (DNA Polymerase I), dNTP's, buffer
d) Purification (Qiaquick Purification Kit)

FIG. 1b
e) PCR, Primer 5′-TGTCATCCTGTTGTCATG, dNTP's, buffer, TAQ
f) Reaction with hydrogen sulfite
g) PCR, primer 1 5′-TGTTATTTTGTTGTTATG, primer 2 5′-TATCATCCTATTATCATA

FIG. 2:
a) Reaction with hydrogen sulfite
b) PCR, primer 1 5′-TGTTATTTTGTTGTTATG, primer 2 5′-TATCATCCTATTATCATA

1. A method for providing demethylated DNA as reference material for the analysis of cytosine methylations in genomic DNA samples with the use of complex amplifications is hereby characterized in that the following method steps are conducted:
a) a genomic DNA sample is amplified, wherein either very short or degenerate oligonucleotides or oligonucleotides complementary to adaptors are used each time as primers;
in the latter case, the genomic DNA is cleaved with a restriction enzyme prior to the amplification, and adaptors are ligated to the ends of the DNA fragments that are formed;
b) the amplificates are chemically treated in such a way that cytosine bases that are unmethylated are converted to uracil, or another base unlike cytosine in hybridization behavior, while the 5-methylcytosine bases remain essentially unchanged;
c) the chemically pretreated amplificates are again amplified, whereby either several specifically hybridizing oligonucleotides or oligonucleotides complementary to the adaptors are used each time as primers; in the latter case, the adaptors are also converted according to the rules of step 1b.

2. A method for the analysis of cytosine methylations in genomic DNA samples with the use of complex amplifications by means of adaptors, is hereby characterized in that the following method steps are conducted:
a) a genomic DNA sample to be investigated is cleaved by means of a restriction enzyme;
b) adaptors are ligated to the ends of the DNA fragments and the sample is then divided; the first portion of the sample is amplified by means of oligonucleotides, which are complementary to the adaptors, as primers, whereas the second portion of the sample is not amplified;
b) the two portions of the sample are chemically treated separately in such a way that cytosine bases that are unmethylated are converted to uracil, or another base unlike cytosine in hybridization behavior, while the 5-methylcytosine bases remain essentially unchanged;
d) the two portions of the sample that are chemically treated are amplified, whereby oligonucleotides complementary to the adaptors after the chemical treatment are used as primers;
e) both portions of the sample are analyzed, whereby the first portion of the sample supplies the reference value for a methylation degree of 0%, and the second portion of the sample supplies the measurement value, which essentially corresponds to the degree of methylation in the original genomic DNA sample.

3. The method according to claim 1 or 2, further characterized in that a PCR (polymerase chain reaction) is used for the amplification.

4. The method according to claim 1 or 2, further characterized in that a heat-stable DNA polymerase is used for the polymerase chain reaction.

5. The method according to claim 1 or 2, further characterized in that the amplification of several DNA segments is conducted in one reaction vessel.

6. The method according to claim 1 or 2, further characterized in that the chemical treatment is conducted with sodium bisulfite (=hydrogen sulfite, disulfite).

7. The method according to claim 1 or 2, further characterized in that after the chemical treatment, the amplificates are separated from reagents and other components of the reaction mixture by binding to a solid phase or to a gel and by washing steps.

8. The method according to claim 1 or 2, further characterized in that after the chemical treatment, the reagents and the other components of the reaction mixture are preferably then diluted in such a way that they are no longer troublesome in the subsequent amplification, but the concentration of the treated amplificate is still sufficient for the second amplification.

9. The method according to claim 1 or 2, further characterized in that one of the following restriction endonucleases is used: RsaI, DpnI, DpnII, MseI, Sau3AI, AluI, NlaIII, HaeIII, BfaI, Tsp509I, BstUI or MboI.

10. The method according to claim 1 or 2, further characterized in that the demethylated reference DNA which is produced is analyzed in the same way as a sample DNA to be investigated and supplies in the analysis the reference value for a methylation degree of 0%.

11. The method according to claim 1 or 2, further characterized in that a DNA which is methylated enzymatically and which is treated in the same way as the sample DNA in the following steps, additionally is used as a reference for a methylation degree of 100%.
